(19) United States
(12) Patent Application Publication
(10) Pub. No.: US 2024/0285450 A1
(43) Pub. Date: Aug. 29, 2024

(54) Portable and Insertable Flatulence Silencer and Gas Deodorizer

(71) Applicant: James Adams, El Cajon, CA (US)

(72) Inventor: James Adams, El Cajon, CA (US)

(21) Appl. No.: 18/174,631

(22) Filed: Feb. 26, 2023

(51) Int. Cl. A61F 13/84

(52) U.S. Cl. CPC A61F 13/8405; A61F 2013/15138; A61F 2013/8408; A61F 2013/8482

(57) ABSTRACT

An ergonomic and novel assembly of a Portable and Insertable Flatulence Silencer and Gas Deodorizer that simultaneously eliminates the sound and smell of flatulence. The apparatus is easy to insert, receives gas and removes bad odors, then releases the gas into the outer environment silently. The apparatus can also be connected to a paired smartphone application or computing device to provide users with real-time information regarding the status of their personal health, including feces and gas.

### COPYRIGHT NOTICE

A portion of the disclosure of this patent document contains material which is subject to copyright protection. The copyright owner has no objection to the facsimile reproduction by anyone of the patient disclosure, as it appears in the Patent and Trademark Office patent files or records, but otherwise reserves all copyright rights whatsoever.

### BACKGROUND

### Field of the Invention

Embodiments disclosed herein generally relate to methods, apparatus, and systems to the field of intestinal discharge control products and more particularly relates to a Portable and Insertable Flatulence Silencer and Gas Deodorizer that simultaneously eliminates the sound and smell of flatulence.

### Description of the Related Art

The average person produces two to three pints of gas daily, which leaves the body in the form of belching or flatulence. On average, a flatulence outburst for a normal person ranges from 10 to 20 occurrences per day. However, sufferers of Irritable Bowel Syndrome, Diabetes, Diverticulitis and other gastrointestinal ailments can produce far more gas, with more odors and emit it more frequently. To minimize the effects of flatulence, a portable flatulence filter seat cushion incorporating replaceable odor and sound filtration was invented. The said cushions are typically used at home, in office environments, for wheelchair patients, while traveling in the car, train, plane, and sporting events or for that special gift-giving occasion.

There are other devices for protecting against or filtering and/or capturing flatulence. Typical of these is U.S. Pat. No. 4,182,335 issued to C. James Matrullo on Jan. 8, 1980.

Another patent was issued to Randolph E. Campbell on Dec. 25, 1984 as U.S. Pat. No. 4,490,145. Yet another U.S. Pat. No. 4,880,417 was issued to Alexander A. Yabrov on Nov. 14, 1989 and still yet another was issued on Nov. 1, 1994 to David W. Revelle as U.S. Pat. No. 5,360,421.

Another patent was issued to Kenneth J Grosse on Sep. 9, 1997 as U.S. Pat. No. 5,665,081. Yet another U.S. Pat. No. 5,728,080 was issued to Masuhiro Suyama on Mar. 17, 1998. Another was issued to Barbara Oakley Sauer on Sep. 28, 1999 as U.S. Pat. No. 5,957,907.

U.S. Pat. No. 4,182,335

### Inventor: C. James Matrullo

### Issued: Jan. 8, 1980

An anal filter for protecting underwear from soiling, is attached to a person's anus solely be non-adhesive adherence to the anus and adjacent portions of the buttocks, due to a facing of fluffy fibrous material, and when attached, serves as a filter by being pervious to the discharge of gas through the anus but substantially impervious to complete passage of excreta carried by the gas or remaining on the anus from a bowel movement. A layer may be adhesively secured to the fibrous material and which prior to use of the anal filter can be pulled off from the fibrous material while pulling this material's fibers outwardly so as to give the material its fluffy characteristic.

U.S. Pat. No. 4,490,145

### Inventor: Randolph E. Campbell

### Issued: Dec. 25, 1984

An ostomy pouch having a filter element affixed to the outer pouch wall. The outer pouch wall has an aperture and the filter element includes a polymeric film cover and an insert of gas deodorizing material. The film cover also has an aperture and opposite ends of the insert overlie the two apertures.

U.S. Pat. No. 5,665,081

### Inventor: Kenneth J. Grosse

### Issued: Sep. 9, 1997

A pad for attenuating sound and absorbing odors from an anal region of an individual. The inventive device includes a pad member containing a volume of odor-absorbing granulated charcoal covered by a layer of filler material and encapsulated with a flexible web. A gluteus insert extends from the pad for positioning between the gluteus-maximus muscles and against the anal region of the user to attenuate noise emanating therefrom.

U.S. Pat. No. 4,880,417

### Inventor: Alexander A. Yabrov

### Issued: Nov. 14, 1989

An anal pad for preventing soiling of underwear due to undesired anal discharge. The anal pad includes a thin outer shell enclosing a semi-rigid shell. The semi-rigid shell may be a layer on one interior surface of the outer shell and may also partially cover the opposite interior surface of the outer shell. An absorbing layer is located within the outer shell and the semi-rigid layer. The absorbing layer contains glycerin which absorbs hydrogen sulfide gas and the combination of the absorbing layer and the semi-rigid shell acts as a sound muffling camera so as to decrease the noise due to flatulence. The absorbing layer is made of a light porous material, the semi-rigid layer is made of compressed paper and the outer shell is made of a thin sheet of lignin paper. A layer of glue is provided on the outer surface of the anal pad for attaching it to a user's underwear.

U.S. Pat. No. 5,360,421

### Inventor: David W. Revelle

### Issued: Nov. 1, 1994

A washable and reusable absorbent garment designed to be fitted about the waist and legs and covering the groin and anal areas of a wearer. The garment includes a number of superimposed layers of textile material sewn together along their edges. Elastic material may be sewn in the material around the leg openings and waist to improve the fit. A liner material covers the inner surface of the garment. The liner layer of the garment or that portion which is next to the wearer's skin is a knitted bicomponent textile comprised of a brushed polyester outer layer which is interknitted with a nylon block co-polymer inner layer. The materials function to transfer moisture in both vapor and liquid form from the polyester side to the nylon side where it is dispersed and made available to the garment's second or absorbent layer. The two specific materials in the liner adjacent the wearer's skin and absorbent material work together to maintain a high degree of comfort for the wearer even after it has been wetted. The structural and functional integrity of the garment is maintained after repeated washings.

U.S. Pat. No. 5,728,080

### Inventor: Masuhiro Suyama

### Issued: Mar. 17, 1998

The present invention relates to a degassing device for an artificial anal bag which is capable of storing the gas generated in the intestines, and of discharging the gas from the bag at a selected place where degassing causes no nuisance to others. It comprises an elongated tube having a filter loaded therein, an opening-and-closing plug attached to its head end and a diverging joint formed at its tail end. The diverging joint can be attached to the circumference of a joint opening, which is made in the upper part of the bag. When the bag is inflated with the gas and excreta from the intestines, the patient can go to the men's room or somewhere for discharging the gas from the bag. Then, the excreta and liquid content are prevented from passing through the filter, only permitting the gas to flow therethrough. Thus, degassing can be put under the perfect control by the patient, so that there is no fear of permitting leakage of gas to cause nuisance to others or imprignation of his underwear with unpleasing odor before the patient is aware.

### SUMMARY

Multiple inventions have been proposed regarding bringing an advancement and enhanced utility for people. As such, considering the foregoing, it may be appreciated that there continues to be a need for novel and improved devices and methods for managing intestinal discharges effectively. The current invention provides a Portable and Insertable Flatulence Silencer and Gas Deodorizer. It simultaneously eliminates the sound and smell of flatulence. The device is inserted into the anus. Gas enters through vent ports in the head of the device, then passes through a hollow tube that contains a gas deodorizing air filter. Bad odors—including sulfur—are absorbed by the filter or covered up by scents contained in the filter. Gas exits the device (and the user's body) silently and without bad odors.

None of the previous inventions and patents, taken either singly or in combination, describe the invention as claimed. Hence, the inventor of the present invention proposes to resolve and surmount existent technical difficulties to eliminate the aforementioned shortcomings of prior art.

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

It is a general objective of the present invention to provide an apparatus that relates to a novel Portable and Insertable Flatulence Silencer and Gas Deodorizer with optional smart device connectivity providing real-time personal health data and alerts to users.

Another objective is to provide an improved, convenient and safe apparatus that promotes ease for the consumer while using the apparatus within their body.

Another objective is to disclose a method to introduce a device that is inserted into the anus and gas enters through vent ports in the head of the device and then passes through a hollow tube that contains a gas-deodorizing air filter. Bad odors, including sulfur, are absorbed by the filter. Gas exits the device and the user's body silently and without bad odors.

It is also the objective of the invention to provide a smart connected health device that can be paired with a smartphone app to notify users in real time of important information related to their personal health, including the status of their feces and gas.

More specifically, it is the objective of this invention to provide an architecture that provides efficient means of providing users with real-time information regarding the status of their feces and gas.

Other aspects, advantages and novel features of the present invention will become apparent from the detailed description of the invention when considered in conjunction with the accompanying drawings.

This summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated into and form part of the specification, and serve to further illustrate embodiments of concepts that include the claimed invention, and explain various principles and advantages of those embodiments.

FIG. 1 depicts a complete view of a device in accordance with one or more embodiments of the invention.

FIG. 2 depicts a complete view of a smart connected device in accordance with one or more embodiments of the invention.

FIG. 3 depicts a filter assembly in accordance with one or more embodiments of the invention.

The apparatus and method components have been represented where appropriate by conventional symbols in the drawings, showing only those specific details.

### DETAILED DESCRIPTION

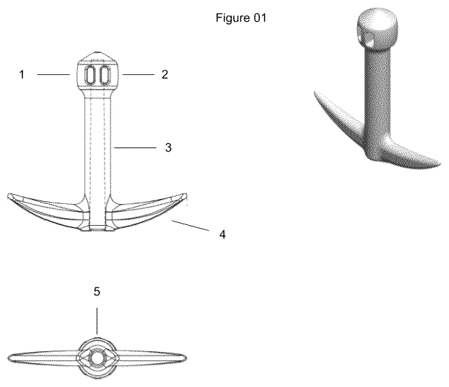

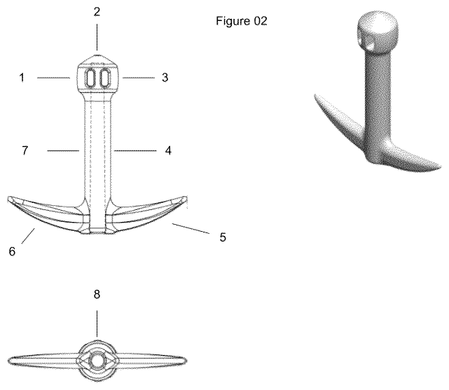

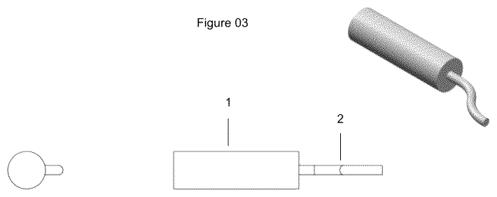

With reference now to the drawings, and in particular to FIGS. 1 through 3 thereof, a new portable and insertable flatulence filter with gas deodorizing air filter embodying the principles and concepts of embodiments of the disclosure will be described. As best illustrated in FIG. 1, the apparatus generally comprises a bulbous head 1 having one or more gas intake ports 2, a hollow tube body 3, a handle 4, and a hole for the insertion and removal of a gas deodorizing air filter 5. As best illustrated in FIG. 2, a smart connected embodiment of the apparatus generally comprises a bulbous head 1 having one or more sensors 2, one or more gas intake ports 3, a hollow tube body 4, a handle 5, a battery, wireless radio chip (e.g. Bluetooth), and vibrator 6, one or more sensors in the body 7, and a hole for the insertion and removal of a gas deodorizing air filter 8. As best illustrated in FIG. 3, the apparatus comprises a gas deodorizing air filter 1 that may or may not include a string to facilitate easy removal from the body of the apparatus 2.

Detailed descriptions of preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention as described provides an apparatus that eliminates the sound and smell of flatulence simultaneously.

In the following, we describe the structure of one embodiment of the Portable and Insertable Flatulence Silencer and Gas Deodorizer which is developed with high-quality materials to provide ease of use, efficiency, and effectiveness to users.

In one embodiment, the apparatus structure as shown in FIG. 1 includes a main body that has a first end designed to be a bulbous head, and a second end designed to be a handle. The head and handle are joined together by a hollow tube body. Within the head are gas intake ports.

A gas deodorizing air filter as per its further embodiments includes a cylindrical body with or without an attached string. In the working apparatus, gas enters through vent ports in the head of the device, then passes through a hollow tube body that contains a gas deodorizing air filter. Bad odors are absorbed by the filter or covered up by scents contained in the filter. Gas exits the device and the user's body silently and without bad odors.

In one embodiment, as shown in FIG. 3, the gas deodorizing filter is easy for users to remove and replace as needed. To facilitate removal, the filter includes a string that protrudes from the bottom of the device. The string makes it easy for users to grab and remove the filter with their fingers. Once a filter is removed, a new filter can be inserted into the device's hollow tube.

As per further embodiments of the invention, the invention may include a scented filter including but not limited to mint or potpourri scent, or a filter that contains odor-absorbing chemicals including but not limited to hydroxypropyl beta-cyclodextrin [HPβCD], the active ingredient in Febreze.

The air filter as per further embodiments may use any of the following deodorizing methods, or a combination of them:
1) Physical absorption of sulfur gas and other bad odors using including but not limited to activated carbon/charcoal;
2) Chemical absorption of sulfur gas and other bad odors using including but not limited to hydroxypropyl beta-cyclodextrin (HPBCD);
3) Natural or artificial scents including but not limited to mint, potpourri, or bubble gum.

In another embodiment, the Portable and Insertable Flatulence Silencer and Gas Deodorizer provides smartphone connectivity with the help of inbuilt sensors, a battery, and a wireless radio chip, e.g. bluetooth low energy. This embodiment is a smart connected health device that can be paired with a smartphone app to notify users of important information such as:
1) When they need to have a bowel movement because their colon is full of feces.
2) When they are constipated.
3) The density of their feces.
4) The water content % of their feces.
5) The volume of gas they are passing.
6) The chemical composition of the gas, including the percentage of sulfur.

This personal health data can be used by a smartphone app to make timely recommendations that will improve the health of users. The data can be viewed and used from a smartphone, computer, laptop, PDA or any computing device. In order to use a connected application, users must wirelessly connect their smartphone or computing device to the invention via Bluetooth or other wireless technology to use the application in real-time.

Generally, client devices may include virtually any computing device capable of receiving and sending a message over a network, such as LAN/WAN/PAN or independent network, wireless network, and the like, to and from another computing device, such as a server or mobile device, and the like. The set of such devices may include devices that typically connect using wired communication medium such as personal computers, multiprocessor systems, microprocessor-based or programmable consumer electronics, network PCs, and the like. The set of such devices may also include devices that typically connect using a wireless communications medium such as cell phones, smart phones, Tablets or PDAs. Similarly, client device may be any device that is capable of connecting using a wired or wireless communication medium such as a PDA, POCKET PC, wearable computer, and any other device that is equipped to communicate over a wired and/or wireless communication medium.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A Portable and Insertable Flatulence Silencer and Gas Deodorizer, comprising:
a. a bulbous head;
b. a hollow tube body;
c. a handle;
d. one or more gas intake ports; and,
e. a gas deodorizing air filter.

2. A Portable and Insertable Flatulence Silencer and Gas Deodorizer as claimed in claim 1, wherein it can be used to eliminate the sound and smell of flatulence.

3. A Portable and Insertable Flatulence Silencer and Gas Deodorizer as claimed in claim 1, wherein a bulbous head is inserted into the anus.

4. A Portable and Insertable Flatulence Silencer and Gas Deodorizer as claimed in claim 1, wherein gas enters through vent ports in the head of the device, then passes through a hollow tube body that contains a gas deodorizing air filter.

5. A Portable and Insertable Flatulence Silencer and Gas Deodorizer as claimed in claim 1, wherein bad odors are absorbed by a gas deodorizing air filter, or covered up by natural or artificial scents contained in the filter.

6. A Portable and Insertable Flatulence Silencer and Gas Deodorizer as claimed in claim 1, wherein gas exits the device and the user's body silently and without bad odors.

2. A Portable and Insertable Flatulence Silencer and Gas Deodorizer with the capability and mechanisms to collect, store, transmit, and receive data, and communicate with smart connected devices and applications, wherein:

7. A Portable and Insertable Flatulence Silencer and Gas Deodorizer as claimed in claim 2, wherein the apparatus comprises one or more sensors, a battery, a wireless radio chip (e.g. Bluetooth), and a vibrator.

8. A Portable and Insertable Flatulence Silencer and Gas Deodorizer as claimed in claim 2, wherein personal health data is collected and transmitted to paired devices and application, and said applications analyze the data, then provide real-time notifications to users via application alerts, or via kinetic tactile alerts facilitated by a vibrator contained in the apparatus.

9. A Portable and Insertable Flatulence Silencer and Gas Deodorizer as claimed in claim 2, wherein personal health data can include but is not limited to data on feces, the presence of feces, the density of feces, the water content of feces, volume of gas, and chemical composition of gas.
